(19) United States
(12) Patent Application Publication
(10) Pub. No.: US 2023/0210228 A1
(43) Pub. Date: Jul. 6, 2023

(54) COOLING BRACELET

(71) Applicant: Fourstar Group Inc., Taipei (TW)

(72) Inventor: Qin Yao, Wuhan (CN)

(73) Assignee: Fourstar Group Inc., Taipei (TW)

(21) Appl. No.: 17/585,892

(22) Filed: Jan. 27, 2022

(30) Foreign Application Priority Data
Dec. 30, 2021 (CN) 202130870354.2

(51) Int. Cl. A44C 5/00

(52) U.S. Cl. CPC A44C 5/0023; A61F 2007/0228

(57) ABSTRACT

A cooling bracelet that is adapted to be worn by a user for the purpose of cooling the wrist area of the user so as to lower the users body temperature after a period of exercise. The cooling bracelet is adapted for use in combination with a cooling pack that is mounted in the cooling bracelet. The cooling bracelet includes a middle pad and separate elongated bracelet straps that extend from the middle pad. Respective securing members are disposed at free ends of each elongated bracelet strap. The respective securing members are for fastening the cooling bracelet about the wrist area of the user. The middle pad of the cooling bracelet has a recess for receiving the cooling pack therein, said recess positioned, when the cooling bracelet is fastened about the wrist area of the user, to face the wrist area of the user in order to provide a cooling effect from the cooling pack and against the wrist area of the user.

### TECHNICAL FIELD

The present invention relates to an improved cooling bracelet that is adapted to be worn by a user for the purpose of cooling the wrist area of the user so as to lower the user's body temperature after a period of exercise. This invention also relates to a cooling bracelet that is simple in construction and yet effective in providing the desired cooling effect. Accordingly, it is an object of the present invention to provide an improved cooling bracelet that is simple in construction, that is effective in its operation and that can be manufactured inexpensively.

### SUMMARY OF THE INVENTION

To accomplish the foregoing and other objects and features of the present invention there is provided a cooling bracelet that is adapted to be worn by a user for the purpose of cooling the wrist area of the user so as to lower the user's body temperature after a period of exercise. The cooling bracelet is adapted for use in combination with a cooling pack that is mounted in the cooling bracelet. The cooling bracelet includes a middle pad and separate elongated bracelet straps that extend from the middle pad. Respective securing members are disposed at free ends of each elongated bracelet strap. The respective securing members are for fastening the cooling bracelet about the wrist area of the user. The middle pad of the cooling bracelet has a recess for receiving the cooling pack therein, said recess positioned, when the cooling bracelet is fastened about the wrist area of the user, to face the wrist area of the user in order to provide a cooling effect from the cooling pack and against the wrist area of the user.

In accordance with other aspects of the present invention one of the respective securing members includes a series of spaced apart holes; another of the respective securing members includes a post for engaging one of the series of spaced apart holes; the middle pad is greater in height than a thickness of either of the separate elongated bracelet straps; the cooling bracelet is constructed of a pliable rubber like material; the recess in the middle pad has a peripheral ledge that extends about sides of the recess; the peripheral ledge provides an interlock with the cooling pack; the cooling pack has a peripheral indentation that engages with the peripheral ledge at the recess; the cooling pack may be rectangular having the indentation formed on all sides of the rectangular cooling pack; and the indentations in the cooling pack form an exposed flat contact surface that is meant to contact the wrist area of the user.

In accordance with the present invention there is also provided a cooling bracelet that is adapted to be worn by a user for the purpose of cooling the wrist area of the user so as to lower the users body temperature after a period of exercise. The cooling bracelet is adapted for use in combination with a cooling pack that is mounted in the cooling bracelet. The cooling bracelet includes a middle pad and separate elongated bracelet straps that extend from the middle pad. Respective securing members are disposed at free ends of each elongated bracelet strap. The respective securing members are for fastening the cooling bracelet about the wrist area of the user. The middle pad of the cooling bracelet has a recess for receiving the cooling pack therein, said recess positioned, when the cooling bracelet is fastened about the wrist area of the user, to face the wrist area of the user in order to provide a cooling effect from the cooling pack and against the wrist area of the user, and respective interlocking segments of the cooling pack and middle pad recess, said cooling bracelet being constructed of a soft pliable material that can give when engaging the cooling pack into the middle pad recess.

In accordance with other aspects of the present invention one of the respective securing members includes a series of spaced apart holes; another of the respective securing members includes a post for engaging one of the series of spaced apart holes; the middle pad is greater in height than a thickness of either of the separate elongated bracelet straps; the respective interlocking segments of the cooling pack and middle pad recess forms a concave to convex interface; the recess in the middle pad has a peripheral ledge that extends about sides of the recess, and that forms the convex surface; the peripheral ledge provides an interlock with the cooling pack; the cooling pack has a peripheral indentation that engages with the peripheral ledge at the recess, and forms the concave surface; and wherein the cooling pack is rectangular having the indentation formed on all sides of the rectangular cooling pack, and the indentations in the cooling pack form an exposed flat contact surface that is meant to contact the wrist area of the user.

In accordance with the present invention there is also provided a method of applying a cooling bracelet that is adapted to be worn by a user for the purpose of cooling the wrist area of the user so as to lower the users body temperature after a period of exercise. The method comprises providing the cooling bracelet adapted for use in combination with a cooling pack; mounting the cooling pack into the cooling bracelet, said cooling bracelet including a middle pad and separate elongated bracelet straps that extend from the middle pad, respective securing members disposed at free ends of each elongated bracelet strap, using the respective securing members for fastening the cooling bracelet about the wrist area of the user; providing the middle pad of the cooling bracelet with a recess for receiving the cooling pack therein, said recess positioned, when the cooling bracelet is fastened about the wrist area of the user, to face the wrist area of the user in order to provide a cooling effect from the cooling pack and against the wrist area of the user; and when inserting the cooling pack into the recess, providing respective interlocking segments of the cooling pack and middle pad recess, said cooling bracelet being constructed of a soft pliable material that can give when engaging the cooling pack into the middle pad recess.

### BRIEF DESCRIPTION OF THE DRAWINGS

It should be understood that the drawings are provided for the purpose of illustration only and are not intended to define the limits of the disclosure. The foregoing and other objects and advantages of the embodiments described herein will become apparent with reference to the following detailed description when taken in conjunction with the accompanying drawings in which:

FIG. 1 is a perspective view of the cooling bracelet of the present invention illustrated on the wrist;

FIG. 2 is a top perspective view of the cooling bracelet;

FIG. 3 is a bottom perspective view of the cooling bracelet;

FIG. 4 is a top view of the cooling bracelet;

FIG. 5 is a cross-sectional view of the cooling bracelet taken along line 5-5 of FIG. 4;

FIG. 6 is a cross-sectional enlarged view of the cooling bracelet;

FIG. 7 is a cross-sectional view of the cooling bracelet taken along line 7-7 of FIG. 6;

FIG. 8 is an exploded view of the cooling bracelet; and

FIG. 9 is a perspective view of the cooling pack.

### DETAILED DESCRIPTION

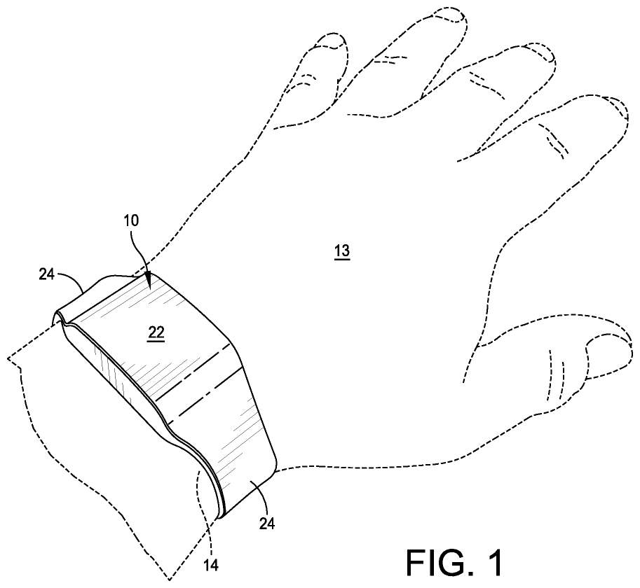

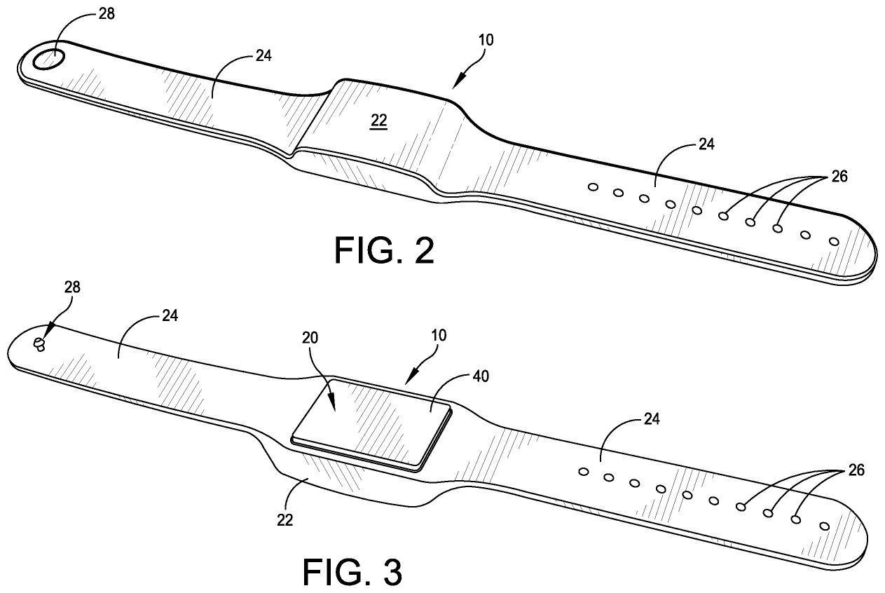

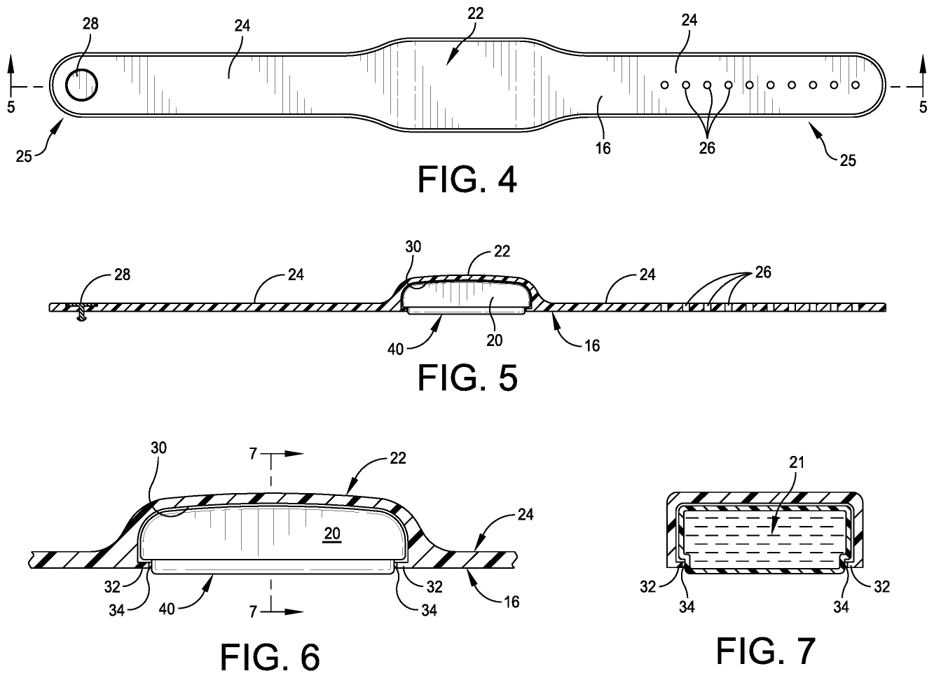

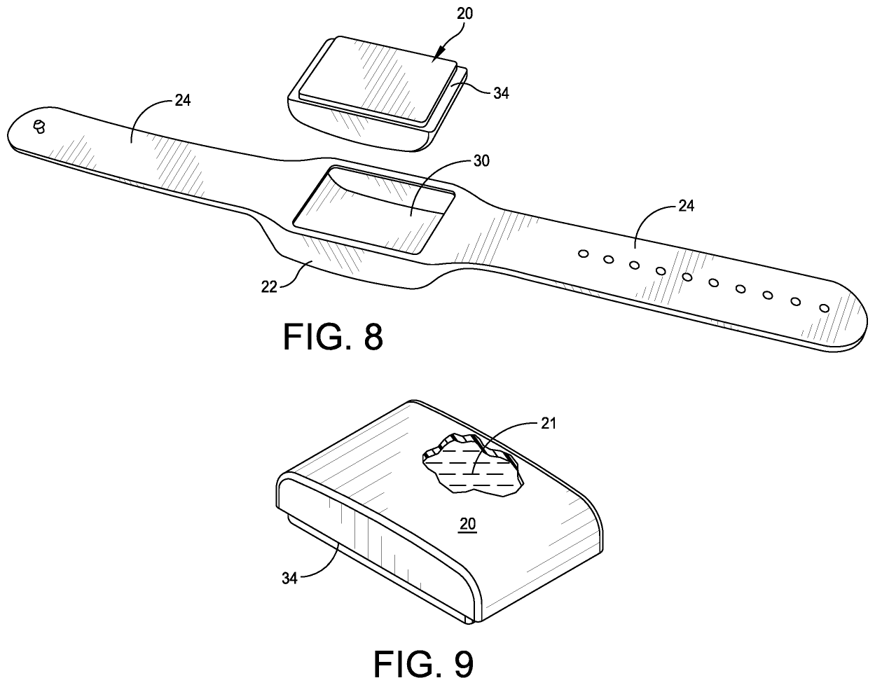

In accordance with the present invention reference is now made to FIGS. 1-9 that illustrate a preferred embodiment of the cooling bracelet 10. The cooling bracelet is adapted to be worn by a user for the purpose of cooling the wrist area 14 of the user 12 so as to lower the user's body temperature after a period of exercise. The cooling bracelet 10 adapted for use in combination with a cooling pack 20 that is mounted in the cooling bracelet 10. The cooling bracelet including a middle pad 22 and separate elongated bracelet straps 24 that extend from the middle pad 22. The middle pad and straps are preferably constructed of a soft rubbery material that is pliable; and is formed in a one-piece arrangement. This soft pliable material permits the cooling pack to be easily engaged with the middle pad recess 30. The cooling pack 20 can simply be slid into the recess 30 and arranged to interlock with the recess as described hereinafter.

Respective securing members 25 are disposed at free ends of each elongated bracelet strap 24. The respective securing members are for fastening the cooling bracelet 10 about the wrist area 14 of the user 12. The middle pad 22 of the cooling bracelet has a recess 30 for receiving the cooling pack 20 therein. The recess 30 is positioned, when the cooling bracelet is fastened about the wrist area of the user, to face the wrist area 14 of the user in order to provide a cooling effect from the cooling pack 20 and against the wrist area 14 of the user 12.

In accordance with other aspects of the present invention one of the respective securing members 25 includes a series of spaced apart holes 26. Another of the respective securing members 25 includes a post 28 for engaging one of the series of spaced apart holes 26. The series of spaced apart holes enables a wide variation in wrist size to be accommodated. In an alternate embodiment of the invention other securing members may be provided such as a hook and loop arrangement or many other means. As shown, for example, in FIG. 5 the middle pad 22 is greater in height than a thickness of either of the separate elongated bracelet straps 24. This provides a sufficient space in the recess 30 for receiving the cooling pack 20.

The recess 30 in the middle pad 22 has a peripheral ledge 32 that preferably extends about all sides of the recess 30. Refer in particular to the cross-sectional views of FIGS. 6 and 7. The peripheral ledge 32 provides an interlock surface with the cooling pack 20 in order to hold the cooling pack in place securely within the recess 30 of the middle pad 22. To provide this interlock the cooling pack 20 has a peripheral indentation 34 that engages with the peripheral ledge 32 at the recess 30. In the illustrated embodiment, the cooling pack 20 is rectangular having the indentation 34 formed on all sides of the rectangular cooling pack 20. The cooling pack 20 may also be constructed in other shapes, but will also be provided with an indentation in order to interlock with the middle pad 22. The indentations 34 in the cooling pack 20 form an exposed flat contact surface 40 that is meant to make direct contact with the wrist area 14 of the user 12.

In accordance with the present invention the interlocking may be by means of respective interlocking segments of the cooling pack 20 and middle pad recess 30. The respective interlocking segments of the cooling 20 and middle pad recess 30 may be considered as formed as a concave to convex interface. This concave/convex arrangement is embodied in the middle pad 22 having the peripheral ledge 32 that extends about sides of the recess 30, and that forms the convex surface. The peripheral ledge 32 provides an interlock with the cooling pack. The cooling pack 20 has a peripheral indentation 34 that engages with the peripheral ledge 32 at the recess, and thus forms the concave surface.

With further reference to FIGS. 6 and 7, another important relationship is having the exposed flat contact surface 40 raised slightly from the bracelet undersurface 16. The exposed flat contact surface 40 may be raised on the order of 1/16 to 1/32 inch. Thus, when the bracelet is in place about the wrist area 14, and secured in place by the securing member 25, there is a force imposed by the securing of the bracelet between the cooling pack surface 40 and the wrist area of the user or wearer. This provides an excellent contact between the cooling pack and the user's wrist. Also, see FIG. 9 where there is an illustration of the use of some type of coolant 2 within the cooling pack 20. One or more of the cooling packs may be left in a refrigerator ready for use with the bracelet.

In accordance with another aspect of the present invention there is a method of applying a cooling bracelet 10 that is adapted to be worn by a user for the purpose of cooling the wrist area of the user so as to lower the user's body temperature after a period of exercise. The method comprises providing the cooling bracelet 10 adapted for use in combination with a cooling pack 20; mounting the cooling pack 20 into the cooling bracelet 10. The cooling bracelet including a middle pad 22 and separate elongated bracelet straps 24 that extend from the middle pad, respective securing members 25 disposed at free ends of each elongated bracelet strap 24. The method further includes using the respective securing members for fastening the cooling bracelet 10 about the wrist area 14 of the user 12, providing the middle pad 22 of the cooling 10 with a recess 30 for receiving the cooling pack therein. The recess 30 is positioned, when the cooling bracelet is fastened about the wrist area of the user, to face the wrist area 14 of the user 12 in order to provide a cooling effect from the cooling pack 20 directly against the wrist 14 of the user 12, when inserting the cooling pack into the recess; and providing respective interlocking segments of the cooling pack and middle pad recess 30. The cooling bracelet 10 is constructed of a soft pliable material that can give when engaging the cooling pack 20 into the middle pad recess 30.

The following is a list of reference numbers that apply.

### REFERENCE NUMBERS

cooling bracelet 10
user 12
users hand 13
wrist area 14
bracelet undersurface 16
cooling pack 20
coolant 21
middle pad 22
elongated bracelet straps 24
respective securing members 25
strap holes 26
strap post
middle pad recess
middle pad ledge
cooling pad peripheral indentation 34
cooling pad flat wrist contact surface 40

Having now described a limited number of embodiments of the present invention, it should now be apparent to those skilled in the art that numerous other embodiments and modifications thereof are contemplated as falling within the scope of the present invention, as defined by the appended claims.

What is claimed is:

1. A cooling bracelet that is adapted to be worn by a user for the purpose of cooling the wrist area of the user so as to lower the users body temperature after a period of exercise, said cooling bracelet adapted for use in combination with a cooling pack that is mounted in the cooling bracelet, said cooling bracelet including a middle pad and separate elongated bracelet straps that extend from the middle pad, respective securing members disposed at free ends of each elongated bracelet strap, said respective securing members for fastening the cooling bracelet about the wrist area of the user, said middle pad of the cooling bracelet having a recess for receiving the cooling pack therein, said recess positioned, when the cooling bracelet is fastened about the wrist area of the user, to face the wrist area of the user in order to provide a cooling effect from the cooling pack and against the wrist area of the user.

2. The cooling bracelet of claim 1 wherein one of the respective securing members includes a series of spaced apart holes.

3. The cooling bracelet of claim 2 wherein another of the respective securing members includes a post for engaging one of the series of spaced apart holes.

4. The cooling bracelet of claim 1 wherein the middle pad is greater in height than a thickness of either of the separate elongated bracelet straps.

5. The cooling bracelet of claim 1 wherein the cooling bracelet is constructed of a pliable rubber like material.

6. The cooling bracelet of claim 1 wherein the recess in the middle pad has a peripheral ledge that extends about sides of the recess.

7. The cooling bracelet of claim 6 wherein the peripheral ledge provides an interlock with the cooling pack.

8. The cooling bracelet of claim 7 wherein the cooling pack has a peripheral indentation that engages with the peripheral ledge at the recess.

9. The cooling bracelet of claim 8 wherein the cooling pack is rectangular having the indentation formed on all sides of the rectangular cooling pack.

10. The cooling bracelet of claim 9 wherein the indentations in the cooling pack form an exposed flat contact surface that is meant to contact the wrist area of the user; and wherein the exposed flat contact surface is raised slightly from a bracelet undersurface.

11. A cooling bracelet that is adapted to be worn by a user for the purpose of cooling the wrist area of the user so as to lower the users body temperature after a period of exercise, said cooling bracelet adapted for use in combination with a cooling pack that is mounted in the cooling bracelet, said cooling bracelet including a middle pad and separate elongated bracelet straps that extend from the middle pad, respective securing members disposed at free ends of each elongated bracelet strap, said respective securing members for fastening the cooling bracelet about the wrist area of the user, said middle pad of the cooling bracelet having a recess for receiving the cooling pack therein, said recess positioned, when the cooling bracelet is fastened about the wrist area of the user, to face the wrist area of the user in order to provide a cooling effect from the cooling pack and against the wrist area of the user, and respective interlocking segments of the cooling pack and middle pad recess, said cooling bracelet being constructed of a soft pliable material that can give when engaging the cooling pack into the middle pad recess.

12. The cooling bracelet of claim 11 wherein one of the respective securing members includes a series of spaced apart holes.

13. The cooling bracelet of claim 12 wherein another of the respective securing members includes a post for engaging one of the series of spaced apart holes.

14. The cooling bracelet of claim 11 wherein the middle pad is greater in height than a thickness of either of the separate elongated bracelet straps.

15. The cooling bracelet of claim 11 wherein the respective interlocking segments of the cooling pack and middle pad recess forms a concave to convex interface.

16. The cooling bracelet of claim 15 wherein the recess in the middle pad has a peripheral ledge that extends about sides of the recess, and that forms the convex surface.

17. The cooling bracelet of claim 16 wherein the peripheral ledge provides an interlock with the cooling pack.

18. The cooling bracelet of claim 17 wherein the cooling pack has a peripheral indentation that engages with the peripheral ledge at the recess, and forms the concave surface.

19. The cooling bracelet of claim 18 wherein the cooling pack is rectangular having the indentation formed on all sides of the rectangular cooling pack, and the indentations in the cooling pack form an exposed flat contact surface that is meant to contact the wrist area of the user.

20. A method of applying a cooling bracelet that is adapted to be worn by a user for the purpose of cooling the wrist area of the user so as to lower the users body temperature after a period of exercise, said method comprising providing the cooling bracelet adapted for use in combination with a cooling pack, mounting the cooling pack into the cooling bracelet, said cooling bracelet including a middle pad and separate elongated bracelet straps that extend from the middle pad, respective securing members disposed at free ends of each elongated bracelet strap, using the respective securing members for fastening the cooling bracelet about the wrist area of the user, providing the middle pad of the cooling bracelet with a recess for receiving the cooling pack therein, said recess positioned, when the cooling bracelet is fastened about the wrist area of the user, to face the wrist area of the user in order to provide a cooling effect from the cooling pack and against the wrist area of the user, when inserting the cooling pack into the recess, providing respective interlocking segments of the cooling pack and middle pad recess, said cooling bracelet being constructed of a soft pliable material that can give when engaging the cooling pack into the middle pad recess.
